(19) United States
(12) Patent Application Publication
(10) Pub. No.: US 2021/0353624 A1
(43) Pub. Date: Nov. 18, 2021

(54) METHOD OF TREATING CANCER WITH TELOTRISAT OR A PRODRUG THEREOF

(71) Applicant: TerSera Therapeutics LLC, Deerfield, IL (US)

(72) Inventors: Suma Gopinathan, Conroe, TX (US); Qi Melissa Yang, The Woodlands, TX (US); Praveen Tyle, Spring, TX (US); Alan Wilson, The Woodlands, TX (US)

(21) Appl. No.: 17/317,120

(22) Filed: May 11, 2021

Related U.S. Application Data
(60) Provisional application No. 63/052,444, filed on Jul. 15, 2020.
(60) Provisional application No. 63/024,446, filed on May 13, 2020.

(51) Int. Cl. A61K 31/506; G01N 33/574; A61K 31/337; A61K 33/243; A61K 31/7068; A61P 35/00

(52) U.S. Cl. CPC A61K 31/506; G01N 33/57492; A61K 31/337; G01N 2496/00; A61K 31/7068; A61P 35/00; A61K 33/243

(57) ABSTRACT

Provided herein are methods of treating cancer with a TPH1 inhibitor. In certain embodiments, methods are provided which display remarkable efficacy and bioavailability for the treatment of certain tumors, for example, a neuroendocrine tumor in a human patient. In certain embodiments, the methods comprising treating the patient with telotristat, a telotristat prodrug, or a pharmaceutically acceptable salt thereof, which can be administered either alone or in combination with other anticancer or antidiarrheal agents.

### CROSS-REFERENCE TO RELATED APPLICATION DATA

This application claims the benefit of and priority to Provisional U.S. Patent Application Ser. No. 63/024,446, filed May 13, 2020, titled, METHOD OF TREATING CANCER WITH TELOTRISTRAT ETHYL, and Provisional U.S. Patent Application Ser. No. 63/052,444, filed Jul. 15, 2020, titled, METHOD OF TREATING CANCER WITH TELOTRISTRAT OR A PRODRUG THEREOF, the disclosures of which are incorporated herein in their entireties.

### FIELD

Provided herein are methods for use in the treatment of cancer, for instance cancer with high levels of serotonin. In certain embodiments, methods are provided which display remarkable efficacy and bioavailability for the treatment of certain tumors, for example, a neuroendocrine tumor in a human patient.

### BACKGROUND OF THE INVENTION

Neuroendocrine tumors (NETs) are a group of complex, heterogeneous neoplasms that arise from neuroendocrine cells in the gastrointestinal tract, lungs, pancreas, and various additional endocrine organs. See, e.g., Raphael, M. J. et al., “Principles of diagnosis and management of neuroendocrine tumors,” Canadian Medical Association Journal 2017; 189(10):E398-e404; Hallet, J. et al., “Exploring the rising incidence of neuroendocrine tumors: a population-based analysis of epidemiology, metastatic presentation, and outcomes,” Cancer. 2015; 121(4):589-597. Some NETs are characterized by high levels of serotonin relative to non-cancerous tissue of the corresponding cell type. See, e.g., Alpini, G. et al., “Serotonin metabolism is dysregulated in cholangiocarcinoma, which has implications for tumor growth,” Cancer Res. 2008, 68(22), 9184-93, doi:10.1158/0008-5472.CAN-08-2133.

Biliary tract cancer (BTC), including cholangiocarcinoma (CCA), is a rare and aggressive malignancy with a very poor prognosis. In patients with advanced disease, the current standard of care is systemic chemotherapy with cisplatin (CIS) and gemcitabine (GEM). Clinical trials of targeted therapies have shown marginal survival benefits, owing in large part to the rarity of these malignancies and the heterogeneity among subtypes. In CCA cell lines and human samples, processes responsible for serotonin metabolism were dysregulated, and serotonin synthesis and secretion were increased.

There is a continuing need for compositions and methods targeting these and other types of cancer.

### SUMMARY OF THE INVENTION

The present disclosure is based, in part, on the discovery that telotristat ethyl effectively treats cancer in cell lines, xenograft models, and humans, as shown in the examples below. Accordingly, in one aspect, provided herein are methods of treating cancer in a patient in need thereof, comprising the step of administering to the patient an amount of a tryptophan hydroxylase 1 (TPH1) inhibitor effective to treat the cancer. In certain embodiments, the TPH1 inhibitor is telotristat. In certain embodiments, the TPH1 inhibitor is a telotristat prodrug, such as telotristat ethyl, or a pharmaceutically acceptable salt thereof. In certain embodiments, the TPH1 inhibitor is a hippurate salt of telotristat ethyl.

In another aspect, provided herein are methods useful, for example, for the treatment of a patient with cancer with high levels of serotonin (e.g., a neuroendocrine tumor). In certain embodiments, the methods display remarkable efficacy for the treatment of a cancer that expresses high levels of TPH1 activity relative to reference cells. In certain embodiments, the TPH1 inhibitor reduces the growth rate of a neuroendocrine tumor in a patient.

In another aspect, provided herein are methods of treating cancer in a patient in need thereof, comprising the step of administering to the patient an amount of a tryptophan hydroxylase 1 (TPH1) inhibitor effective to treat the cancer in combination with one or more additional anticancer agents. Exemplary additional anticancer agents include, but are not limited to, gemcitabine, cisplatin, gemcitabine plus cisplatin, paclitaxel, and nab-paclitaxel.

In another aspect, provided herein are methods of treating cancer in a patient in need thereof, comprising the step of administering to the patient an amount of a tryptophan hydroxylase 1 (TPH1) inhibitor effective to treat the cancer in combination with one or more somatostatin analogs. Exemplary somatostatin analogs include, but are not limited to, lanreotide, octreotide, and pasireotide.

### BRIEF DESCRIPTION OF THE FIGURES

FIG. 1 shows the base change in weight for an athymic mouse model upon administration of telotristat ethyl in combination with cancer therapeutics (Example 2).

FIG. 2 provides shows the effect of treatment on tumor size for mice with KKU-M213 tumor line xenographs (Example 2).

FIG. 3 provides the effect of treatment on tumor size for mice with HuCCT1 tumor cell line xenographs (Example 2).

FIGS. 4A and 4B provide dosing in an animal xenograft study (FIG. 4A) as well as percent survival versus time in the same study (Example 3) (FIG. 4B).

FIGS. 5A to 5D show the tumor growth inhibition from telotristat ethyl, gemcitabine+cisplatin, and nab-paclitaxel in an animal xenograft study (Example 3), including the relative tumor volume (FIG. 5A), the net tumor growth (FIG. 5B), the tumor weight (FIG. 5C), and body weight (FIG. 5D).

FIG. 6 provides a graphical summary of physician-reported tumor responses before and after treatment with telotristat ethyl (Example 3).

FIG. 7A illustrates the changes in documented carcinoid syndrome symptoms from medical charts of patients (n=200) after telotristat ethyl initiation (Example 4).

FIG. 7B illustrates the changes in documented carcinoid syndrome symptoms from medical charts of patients in the treatment subgroup (n=65) after telotristat ethyl initiation (Example 4).

FIG. 8A illustrates the changes in body weight for patients from the overall population and from the same treatment subgroup (Example 4).

FIG. 8B illustrates the changes in Eastern Cooperative Oncology Group (ECOG) Performance Status for patients from the overall population and from the same treatment subgroup (Example 4).

### DETAILED DESCRIPTION

Provided herein are compounds, compositions and methods useful for treating cancer in a subject. Further provided are dosage forms useful for such methods.

### Definitions

When referring to the compounds provided herein, the following terms have the following meanings unless indicated otherwise. Unless defined otherwise, all technical and scientific terms used herein have the same meaning as is commonly understood by one of ordinary skill in the art. In the event that there is a plurality of definitions for a term herein, those in this section prevail unless stated otherwise.

“Pharmaceutically acceptable salt” refers to any salt of a compound provided herein which retains its biological properties and which is not toxic or otherwise undesirable for pharmaceutical use. Such salts may be derived from a variety of organic and inorganic counter-ions well known in the art. Such salts include, but are not limited to: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids; or (2) salts formed when an acidic proton present in the parent compound either (a) is replaced by a metal ion, e.g., an alkali metal ion, an alkaline earth ion or an aluminum ion, or alkali metal or alkaline earth metal hydroxides, such as sodium, potassium, calcium, magnesium, aluminum, lithium, zinc, and barium hydroxide, ammonia or (b) coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like.

Pharmaceutically acceptable salts further include, by way of example only and without limitation, sodium, potassium, calcium, magnesium, ammonium, tetraalkylammonium and the like, and when the compound contains a basic functionality, salts of non-toxic organic or inorganic acids, such as hydrohalides, e.g. hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate and the like.

Similarly, the term “isolated” with respect to a composition refers to a composition that includes at least 85, 90%, 95%, 98%, 99% to 100% by weight of the compound, the remainder comprising other chemical species or enantiomers.

As used herein, the terms “subject” and “patient” are used interchangeably herein. The terms “subject” and “subjects” refer to an animal, such as a mammal including a non-primate (e.g., a cow, pig, horse, cat, dog, rat, and mouse) and a primate (e.g., a monkey such as a cynomolgous monkey, a chimpanzee and a human), and for example, a human. In certain embodiments, the subject is refractory or non-responsive to current treatments for hepatitis C infection. In another embodiment, the subject is a farm animal (e.g., a horse, a cow, a pig, etc.) or a pet (e.g., a dog or a cat). In certain embodiments, the subject is a human.

Unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

Unless otherwise indicated, the terms “prevent,” “preventing” and “prevention” contemplate an action that occurs before a patient begins to suffer from the specified disease or disorder, which inhibits or reduces the severity of the disease or disorder. In other words, the terms encompass prophylaxis.

Unless otherwise indicated, a “prophylactically effective amount” of a compound is an amount sufficient to prevent a disease or condition, or one or more symptoms associated with the disease or condition, or to prevent its recurrence. A prophylactically effective amount of a compound means an amount of therapeutic agent, alone or in combination with other agents, which provides a prophylactic benefit in the prevention of the disease or condition. The term “prophylactically effective amount” can encompass an amount that improves overall prophylaxis or enhances the prophylactic efficacy of another prophylactic agent.

Unless otherwise indicated, a “therapeutically effective amount” of a compound is an amount sufficient to provide a therapeutic benefit in the treatment or management of a disease or condition, or to delay or minimize one or more symptoms associated with the disease or condition. A therapeutically effective amount of a compound means an amount of therapeutic agent, alone or in combination with other therapies, which provides a therapeutic benefit in the treatment or management of the disease or condition. The term “therapeutically effective amount” can encompass an amount that improves overall therapy, reduces or avoids symptoms or causes of a disease or condition, or enhances the therapeutic efficacy of another therapeutic agent.

Unless otherwise indicated, the terms “treat,” “treating” and “treatment” contemplate an action that occurs while a patient is suffering from the specified disease or disorder, which reduces the severity of the disease or disorder or one or more of its symptoms, or retards or slows the progression of the disease or disorder.

Unless otherwise indicated, the term “include” has the same meaning as “include, but are not limited to,” and the term “includes” has the same meaning as “includes, but is not limited to.” Similarly, the term “such as” has the same meaning as the term “such as, but not limited to.”

Unless otherwise indicated, the terms “a,” “an,” and “the” not only include aspects and embodiments with one member, but also aspects and embodiments with more than one member. For example, a composition comprising an active agent and an excipient may in certain aspects include at least a second active agent, at least a second excipient, or both.

Unless otherwise indicated, the term “about” as used herein to modify a number indicates that number and a defined range around that number. If “X” were the value, “about X” would generally indicate a value from 0.95X to 1.05X. Any reference to “about X’ specifically indicates at least the values X, 0.95X, 0.96X, 0.97X, 0.98X, 0.99X, 1.01X, 1.02X, 1.03X, 1.04X, and 1.05X. For example, “about 250” is intended to teach and to provide support for a specific claim limitation of, e.g., “238,” “240,” “242,” “245,” “248,” “250,” “252,” “255,” “258,” “260,” and “262.”

Unless otherwise indicated, the term “agent” as used herein indicates a compound or mixture of compounds that produce a particular effect. For example, an “anticancer agent” that is added to a composition provides the composition with therapeutic activity against one or more types of cancer.

Unless otherwise indicated, the term “or” should in general be construed as not excluding the possibility of “and” (i.e., a Boolean “or”). For example, a “composition comprising A or B” would typically present an aspect in which the composition comprised A and B. “Or” should, however, be construed to exclude those aspects presented that cannot be combined without contradiction (e.g., a composition pH that is between 7 and 8 or between 9 and 10).

Unless otherwise indicated, one or more adjectives immediately preceding a series of nouns is to be construed as applying to each of the nouns. For example, the phrase “optionally substituted alkyl, aryl, or heteroaryl” has the same meaning as “optionally substituted alkyl, optionally substituted aryl, or optionally substituted heteroaryl.”

Unless otherwise indicated, a structure or name of a compound or genus of compounds encompasses all forms of that compound or genus of compounds, and all compositions comprising that compound or genus of compounds.

It should also be noted that any atom shown in a drawing with unsatisfied valences is assumed to be attached to enough hydrogen atoms to satisfy the valences. In addition, chemical bonds depicted with one solid line parallel to one dashed line encompass both single and double (e.g., aromatic) bonds, if valences permit. Structures that represent compounds with one or more chiral centers, but which do not indicate stereochemistry (e.g., with bolded or dashed lines), encompasses pure stereoisomers and mixtures (e.g., racemic mixtures) thereof. Similarly, names of compounds having one or more chiral centers that do not specify the stereochemistry of those centers encompass pure stereoisomers and mixtures thereof

Compounds

U.S. Patent Application Publication No. US 2012/0316171 A1 suggested that tryptophan hydroxylase inhibitors, which can reduce the production of serotonin, could be used to treat cancer. However, the application did not provide any data evidencing such an effect that might direct the skilled artisan to specific preferred compounds.

Without intending to be bound by theory, this invention applies the discovery that the TPH1 inhibitor telotristat, prodrugs of telotristat, and pharmaceutically acceptable salts thereof, can affect tumor growth and may consequently be effective in the treatment of some forms of cancer. In certain embodiments, the TPH1 inhibitor is telotristat ethyl, or a pharmaceutically acceptable salt thereof. Telotristat ethyl is (S)-ethyl 2-amino-3-(4-(2-amino-6-((R)-1-(4-chloro-2-(3-methyl-1H-pyrazol-1-yl)phenyl)-2,2,2-trifluoroethoxy)pyrimidin-4-yl)phenyl)propanoate. In certain embodiments, the TPH1 inhibitor is the hippurate salt of telotristat ethyl. The hippurate salt of telotristat ethyl is sold under the tradename XERMELO for the treatment of carcinoid syndrome.

Methods of Treatment

Provided herein are methods of treating cancer with a TPH1 inhibitor. In certain embodiments, the TPH1 inhibitor is any TPH1 inhibitor, or a prodrug or pharmaceutically acceptable salt thereof, that is deemed suitable by the person of skill in the art. The cancer can be any cancer deemed suitable by the practitioner of skill.

In certain embodiments, the TPH1 inhibitor is telotristat, or a pharmaceutically acceptable salt thereof. In certain embodiments, the TPH1 inhibitor is telotristat, a telotristat prodrug (e.g., telotristat ethyl), or a pharmaceutically acceptable salt thereof.

In certain embodiments, the TPH1 inhibitor does not inhibit TPH2 activity. In certain embodiments, the TPH1 inhibitor does not inhibit TPH2 activity in vivo. In certain embodiments, the TPH1 inhibitor inhibits only peripheral TPH1 activity. In certain embodiments, the TPH1 inhibitor inhibits only peripheral TPH1 activity in vivo (e.g., because of an inability to cross the blood-brain barrier).

In certain embodiments, the TPH1 inhibitor reduces the growth rate of a neuroendocrine tumor in a patient. In certain embodiments, the TPH1 inhibitor is administered with one or more anticancer agents to reduce the growth rate of the neuroendocrine tumor. In certain embodiments, the reduction in growth rate reduces the size of a treated neuroendocrine tumor as compared to the size predicted for an untreated tumor (e.g., as compared to the average increase in a control population; as compared to the rate of increase before the treatment period). In certain embodiments, the size is determined by weight, calculated weight, volume, calculated volume, or other physical dimension, such as the sum of the longest diameters of target lesions. In certain embodiments, the tumor size is reduced by at least about 10% (e.g., at least about 10%, 15% 20%, 25%, 30%, 35%, 40%, 45%, 50%, 55%, 60%, 65%, 70%, 75%, or 80%). In certain embodiments, the reduction is over a specified time period (e.g., a 3, 6, 9, 12, 18, or 24-month period).

In certain embodiments, the tumor size is reduced about 45%, 50%, or 55% (e.g., Example 3 TE therapy). In certain embodiments, the tumor size is reduced about 70, 75% or 80% (e.g., Example 3 TE combination therapy). In certain embodiments, the tumor size is reduced at least about 5%, 6%, 7%, 8%, 8.5%, 9%, or 10% (e.g., Example 4).

In certain embodiments, the tumor expresses high levels of TPH1 activity relative to reference cells. The TPH1 activity is identified by identifying the level of TPH1 messenger ribonucleic acid (mRNA) using standard procedures known to the skilled artisan. In certain embodiments, the TPH1 activity is identified by quantitative real-time polymerase chain reaction (RT-PCR). In certain embodiments, the TPH1 activity is identified by Northern blotting. In certain embodiments, the TPH1 activity is identified by a nuclease protection assay. In certain embodiments, the TPH1 activity is identified by an in situ hybridization assay.

In certain embodiments, the method further comprises screening the neuroendocrine tumor for TPH1 mRNA level; and approving the patient for TPH1 inhibitor treatment only when the TPH1 mRNA level of the neuroendocrine tumor is at least 30 times that of the reference cells (e.g., at least 30, 35, 40, 45, 50, or 55 times). In certain embodiments, the TPH1 mRNA level is at least 60 times that of the reference cells (e.g., at least 60, 65, 70, 75, 80, 85, 90, or 95 times). In certain embodiments, the TPH1 mRNA level is at least 100 times that of the reference cells (e.g., at least 100, 125, 150, 175, 200, 225, or 250 times). In certain embodiments, the reference cells are HeLa cells.

Ki-67 is a protein that can be used as a signal of cellular growth, as it appears in the cell nucleus during the active phases of the cell cycle. See, e.g., Gerdes, J. et al. Immunol. 1984, 133, 1710-5. The Ki-67 proliferation index is a measurement of the fraction of cells that are dividing as a percentage of nuclei observed to include Ki-67 from a total sample. See, e.g., Brown, J. R. et al. “Quantitative assessment Ki-67 score for prediction of response to neoadjuvant chemotherapy in breast cancer,” Laboratory Investigation 2014, 94, 98-106. In general, a high Ki-67 index is an indicator of aggressive proliferation for tumor cells, though the threshold for “high” Ki activity may vary, and Ki-67 has been used to evaluate prognosis or therapeutic response in cancer chemotherapy, such as in treatment of breast cancer (e.g., early-stage or preoperative breast cancer). See, e.g., Urruticoechea, A. et al. J. Clin. Oncol. 2005, 23, 7212-20; Varga, Z. et al. Natureresearch: Scientific Reports, 2019, 9:13534, doi.org/10.1038/s41598-019-49638-4.

The Ki-67 proliferation index can be identified by standard methods known to the skilled artisan. In certain aspects, the Ki-67 proliferation index is determined by a pathologist's evaluation of a number of cells (e.g., 2000, 1500, or 1000) from one or more slices (e.g., a 2 mm slice; up to six such slices) from a paraffined block of tumor tissue, which is prepared by standard procedures. The tumor cells are typically stained (e.g., by standard immunohistochemical methods with an anti-Ki-67 antibody).

In certain aspects, the evaluation is conducted by direct assessment of nuclear immunoreactivity by light microscopy (e.g., at 20×, 40×, 100×, 200×, or 400× magnification). In certain aspects, the evaluation of nuclear immunoreactivity is conducted by assessment of computer imaging analysis. In certain aspects, the evaluation is conducted by assessment of randomly chosen fields (e.g., 4, 5, 6, or more fields, each with ca. 250, 400, 500, or more cells). In certain aspects, the fields are from both the periphery and center of the tumor (i.e., “region” analysis). In certain aspects, the fields are from those of the highest Ki activity (i.e., “hotspot” analysis). In certain aspects, the fields are chosen only from the periphery of the tumor and are examined for darkly stained nuclei (i.e., “threshold intensity” analysis). In certain aspects, a combination of methods (e.g., direct assessment, region, and hotspot) was used. See, e.g., Varga, Z. et al. PLoS ONE 10(4): e0123435, doi.org/10.1371/journal.pone.0123435 (providing several standard methods for evaluation of Ki-67 index).

In certain embodiments, the method further comprises screening the neuroendocrine tumor by a Ki-67 index test; and approving the patient for TPH1 inhibitor treatment only when the Ki-67 index is no greater than 20% (e.g., no greater than about 20%, 17.5%, 15%, 12.5%, or 10%, 9%, 8%, 7%, 6%, 5%, 4%, or 3%). In certain embodiments, the instant method comprises approving the patient for TPH1 inhibitor treatment only when the Ki-67 index is no greater than 10% (e.g., no greater than about 10%, 9%, 8%, 7%, 6%, 5%, 4%, or 3%). In certain embodiments, the instant method comprises approving the patient for TPH1 inhibitor treatment only when the Ki-67 index is no greater than 30% (e.g., no greater than about 30%, 29%, 28%, 27%, 26%, 25%, 24%, 23%, 22%, 21%, or 20%).

In certain embodiments, the instant method comprises approving the patient for TPH1 inhibitor treatment only when the Ki-67 index is no greater than 3% (e.g., about 3%, 2.5%, 2%, 1.5%, 1%, 0.75%, or 0.5%). In certain embodiments, the method comprises approving the patient for TPH1 inhibitor treatment only when the Ki-67 index is no greater than 1% (e.g., about 1.0%, 0.9%, 0.8%, 0.7%, 0.6%, 0.5%, 0.4%, 0.3%, 0.2%, or 0.1%).

In certain embodiments, the method further comprises screening the neuroendocrine tumor by a Ki-67 index test; and discontinuing the TPH1 inhibitor treatment when the Ki-67 index is greater than 20% (e.g., greater than about 30%, 29%, 28%, 27%, 26%, 25%, 24%, 23%, 22%, 21%, or 20%). In certain embodiments, the method further comprises discontinuing the TPH1 inhibitor treatment when the Ki-67 index is greater than 10% (e.g., greater than about 20%, 17.5%, 15%, 12.5%, or 10%, 9%, 8%, 7%, 6%, 5%, 4%, or 3%). In certain embodiments, the instant method comprises approving the patient for TPH1 inhibitor treatment only when the Ki-67 index is greater than 1% (e.g., greater than about 10%, 9%, 8%, 7%, 6%, 5%, 4%, 3%, 2%, or 1%).

The Eastern Cooperative Oncology Group (ECOG) Performance status is a method of evaluating a patient's impairment by the progression of his or her disease. It includes the values of 0 (not impaired in activities), 1 (restricted in physically strenuous activity), 2 (ambulatory, but unable to work), 3 (only limited self-care), 4 (completely disabled), and 5 (dead.) See, e.g., ecog-acrin.org/resources/ecog-performance-status. In certain embodiments, the method further comprises screening the patient by an ECOG test; and approving the patient for TPH1 inhibitor treatment only when the ECOG index is no greater than 2 (e.g., 2, 1, or 0).

In certain embodiments, the method further comprises concurrently treating the patient with an antitumor agent. In certain embodiments, the antitumor agent is gemcitabine, cisplatin, paclitaxel, a paclitaxel derivative, or a combinations thereof. In certain embodiments, the antitumor agent is a combination of gemcitabine and cisplatin. In certain embodiments, the antitumor agent is a combination of albumin and paclitaxel. In certain embodiments, the antitumor agent is nab-paclitaxel. Appropriate dosages of the anticancer agents are identifiable from their label information or are otherwise determinable by the skilled artisan by known methods (e.g., by comparison with the exemplary methods herein).

In certain embodiments, the method further comprises concurrently treating the patient with a somatostatin analog (SSA). In certain embodiments, the somatostatin analog is lanreotide, octreotide, pasireotide, or a combination thereof. In certain embodiments, the analog is lanreotide. In certain embodiments, the analog is octreotide. In certain embodiments, the analog is pasireotide. Appropriate dosages of the SSAs are identifiable from their label information or are otherwise determinable by the skilled artisan by known methods (e.g., by comparison with the exemplary methods herein).

In certain embodiments, the cancer is biliary tract cancer, bladder cancer, breast cancer, prostate cancer, small-cell lung cancer, or a combination thereof. In certain embodiments, the cancer is cholangiocarcinoma. In certain embodiments, the patient experiences carcinoid-syndrome-related diarrhea. In certain embodiments, the patient does not experience carcinoid-syndrome-related diarrhea.

In certain embodiments, the method further comprises screening the patient for carcinoid-syndrome-related diarrhea; and approving the patient for TPH1 inhibitor treatment only if the patient has carcinoid-syndrome-related diarrhea. The presence of carcinoid-syndrome-related diarrhea can be diagnosed by methods known to the skilled artisan. See, e.g., American Cancer Society, “Tests for Gastrointestinal Carcinoid Tumors,” available at www.cancer.org/cancer/gastrointestinal-carcinoid-tumor/detection-diagnosis-staging/how-diagnosed.html. In certain embodiments, the screening method for carcinoid-syndrome-related diarrhea is a barium X-ray (e.g., a barium swallow for examination of the esophagus; an upper-GI series with small bowel follow-through; enteroclysis; and a barium enema). In certain embodiments, the screening method for carcinoid-syndrome-related diarrhea is endoscopy (e.g., upper endoscopy, colonoscopy, flexible sigmoidoscopy, capsule endoscopy, double balloon endoscopy, and endoscopic ultrasound). In certain embodiments, the screening method for carcinoid-syndrome-related diarrhea is a computed tomography (CT) scan. In certain embodiments, the screening method for carcinoid-syndrome-related diarrhea is a magnetic resonance imaging (MRI) scan (e.g., MR angiography). In certain embodiments, the screening method for carcinoid-syndrome-related diarrhea is a radionucleotide scan (e.g., a positron emission tomography (PET) scan, somatostatin receptor scintigraphy, or a radioactive iodine (I¹³¹) scan). In certain embodiments, the screening method for carcinoid-syndrome-related diarrhea is a biopsy. In certain embodiments, the screening method for carcinoid-syndrome-related diarrhea is a blood or urine test (e.g., to identify levels of 5-hydroxyindoleacetic acid (5-HIAA)).

The dose can be any dose deemed suitable by the practitioner of skill. In certain embodiments, the TPH1 inhibitor dose is from about 100 to 500 mg (e.g., about 100, 110, 120, 125, 130, 140, 150, 160, 170, 175, 180, 190, 200, 210, 220, 225, 230, 240, 250, 260, 270, 275, 280, 290, 300, 310, 320, 325, 330, 340, 350, 360, 370, 375, 380, 390, 400, 410, 420, 425, 430, 440, 450, 460, 470, 475, 480, 490, or 500 mg). In certain embodiments, the TPH1 inhibitor dose is about 250 mg. In certain embodiments, the TPH1 inhibitor dose is administered from one to five times per day (e.g., 1, 2, 3, 4, or 5). In certain embodiments, the TPH1 inhibitor dose is administered three times per day.

Pharmaceutical Compositions and Methods of Administration

The TPH1 inhibitor can be in any formulation deemed suitable by the practitioner of skill. In certain embodiments, the TPH1 inhibitor is in the form of the commercial pharmaceutical XERMELO. Pharmaceutical compositions and dosage forms of this invention may optionally contain one or more pharmaceutically acceptable carriers or excipients. Certain pharmaceutical compositions are single unit dosage forms suitable for oral, topical, mucosal (e.g., nasal, pulmonary, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The formulation should suit the mode of administration. For example, oral administration may require enteric coatings to protect the active ingredient from degradation within the gastrointestinal tract. In another example, the active ingredient may be administered in a liposomal formulation to shield it from degradative enzymes, facilitate transport in circulatory system, and/or effect delivery across cell membranes to intracellular sites.

The composition, shape, and type of dosage forms of the invention will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18^{th} ed., Mack Publishing, Easton Pa. (1990).

The dosages of the second agents are to be used in the combination therapies provided herein. In certain embodiments, dosages lower than those which have been or are currently being used to prevent or treat cancer are used in the combination therapies provided herein. The recommended dosages of second agents can be obtained from the knowledge of those of skill. For those second agents that are approved for clinical use, recommended dosages are described in, for example, Hardman et al., eds., 1996, Goodman & Gilman's The Pharmacological Basis Of Basis Of Therapeutics 9^{th} Ed, Mc-Graw-Hill, New York; Physician's Desk Reference (PDR) 57^{th} Ed., 2003, Medical Economics Co., Inc., Montvale, N.J., which are incorporated herein by reference in its entirety.

In various embodiments, the therapies (e.g., a compound provided herein and the second agent) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In various embodiments, the therapies are administered no more than 24 hours apart or no more than 48 hours apart. In certain embodiments, two or more therapies are administered within the same patient visit. In other embodiments, the compound provided herein and the second agent are administered concurrently.

In other embodiments, the compound provided herein and the second agent are administered at about 2 to 4 days apart, at about 4 to 6 days apart, at about 1 week apart, at about 1 to 2 weeks apart, or more than 2 weeks apart.

In certain embodiments, administration of the same agent may be repeated, and the administrations may be separated by at least 1 day, 2 days, 3 days, 5 days, 10 days, 15 days, 30 days, 45 days, 2 months, 75 days, 3 months, or 6 months. In other embodiments, administration of the same agent may be repeated, and the administration may be separated by at least at least 1 day, 2 days, 3 days, 5 days, 10 days, 15 days, 30 days, 45 days, 2 months, 75 days, 3 months, or 6 months.

In certain embodiments, a compound provided herein and a second agent are administered to a patient, for example, a mammal, such as a human, in a sequence and within a time interval such that the compound provided herein can act together with the other agent to provide an increased benefit than if they were administered otherwise. For example, the second active agent can be administered at the same time or sequentially in any order at different points in time; however, if not administered at the same time, they should be administered sufficiently close in time so as to provide the desired therapeutic or prophylactic effect. In certain embodiments, the compound provided herein and the second active agent exert their effect at times which overlap. Each second active agent can be administered separately, in any appropriate form and by any suitable route. In other embodiments, the compound provided herein is administered before, concurrently or after administration of the second active agent.

In certain embodiments, the compound provided herein and the second agent are cyclically administered to a patient. Cycling therapy involves the administration of a first agent (e.g., a first prophylactic or therapeutic agents) for a period of time, followed by the administration of a second agent and/or third agent (e.g., a second and/or third prophylactic or therapeutic agents) for a period of time and repeating this sequential administration. Cycling therapy can reduce the development of resistance to one or more of the therapies, avoid or reduce the side effects of one of the therapies, and/or improve the efficacy of the treatment.

In certain embodiments, the compound provided herein and the second active agent are administered in a cycle of less than about 3 weeks, about once every two weeks, about once every 10 days or about once every week. One cycle can comprise the administration of a compound provided herein and the second agent by infusion over about 90 minutes every cycle, about 1 hour every cycle, about 45 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

In other embodiments, courses of treatment are administered concurrently to a patient, i.e., individual doses of the second agent are administered separately yet within a time interval such that the compound provided herein can work together with the second active agent. For example, one component can be administered once per week in combination with the other components that can be administered once every two weeks or once every three weeks. In other words, the dosing regimens are carried out concurrently even if the therapeutics are not administered simultaneously or during the same day.

The second agent can act additively or synergistically with the compound provided herein. In certain embodiments, the compound provided herein is administered concurrently with one or more second agents in the same pharmaceutical composition. In another embodiment, a compound provided herein is administered concurrently with one or more second agents in separate pharmaceutical compositions. In still another embodiment, a compound provided herein is administered prior to or subsequent to administration of a second agent. Also contemplated are administration of a compound provided herein and a second agent by the same or different routes of administration, e.g., oral and parenteral. In certain embodiments, when the compound provided herein is administered concurrently with a second agent that potentially produces adverse side effects including, but not limited to, toxicity, the second active agent can advantageously be administered at a dose that falls below the threshold that the adverse side effect is elicited.

Also provided are kits for use in methods of treatment of cancer that is characterized (e.g., a neuroendocrine tumor). The kits can include a compound or composition provided herein, a second agent or composition, and instructions providing information to a health care provider regarding usage for treating the disorder. Instructions may be provided in printed form or in the form of an electronic medium such as a floppy disc, CD, or DVD, or in the form of a website address where such instructions may be obtained. A unit dose of a compound or composition provided herein, or a second agent or composition, can include a dosage such that when administered to a subject, a therapeutically or prophylactically effective plasma level of the compound or composition can be maintained in the subject for at least 1 days. In some embodiments, a compound or composition can be included as a sterile aqueous pharmaceutical composition or dry powder (e.g., lyophilized) composition.

In some embodiments, suitable packaging is provided. As used herein, “packaging” includes a solid matrix or material customarily used in a system and capable of holding within fixed limits a compound provided herein and/or a second agent suitable for administration to a subject. Such materials include glass and plastic (e.g., polyethylene, polypropylene, and polycarbonate) bottles, vials, paper, plastic, and plastic-foil laminated envelopes and the like. If e-beam sterilization techniques are employed, the packaging should have sufficiently low density to permit sterilization of the contents.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the examples and throughout the specification: g (grams); mg (milligrams); mL (milliliters); μL (microliters); mM (millimolar); μM (micromolar); Hz (Hertz); MHz (megahertz); mmol (millimoles); hr or hrs (hours); min (minutes); MS (mass spectrometry); ESI (electrospray ionization); TLC (thin layer chromatography); HPLC (high pressure liquid chromatography); THF (tetrahydrofuran); CDCl₃ (deuterated chloroform); AcOH (acetic acid); DCM (dichloromethane); DMSO (dimethylsulfoxide); DMSO-d6 (deuterated dimethylsulfoxide); EtOAc (ethyl acetate); MeOH (methanol); and BOC (t-butyloxycarbonyl).

For all of the following examples, standard work-up and purification methods known to those skilled in the art can be utilized. Unless otherwise indicated, all temperatures are expressed in ° C. (degrees Centigrade). All reactions are conducted at room temperature unless otherwise noted. Synthetic methodologies illustrated herein are intended to exemplify the applicable chemistry through the use of specific examples and are not indicative of the scope of the disclosure.

### Example 1

### Telotristat Ethyl is a Potent Inhibitor of Tumor Cell Lines

This example provides experiments assessing the effects of telotristat ethyl on three aggressive cancer cell lines—liposarcoma (94T778), colon cancer (HT-29), and cholangiocarcinoma (TFK-1)—and three “normal” cell lines—fallopian tube epithelium (FT240), ovarian surface epithelium (OSE22), and first trimester placental fibroblasts (FTP).

The cells were grown with 0, 5, 15 and 30 μM telotristat ethyl, plated into four-chambered slides with an initial confluence of at least 30%, and cultured for 24 to 96 hours. At the end of each 24-hour period, cell density was measured with hematoxylin, and the presence of 5-HT was assessed by immunohistochemistry. Experiments were performed in triplicate. Cell confluence was determined, and the 5-HT expression was recorded as an H-score.

In the absence of telotristat ethyl, all the cell lines increased their confluence steadily over the 4 days of culture. When the aggressive cell lines were exposed to increasing concentrations of telotristat ethyl, their confluence decreased progressively, with the 94T778 cells being most impacted, the TFK-1 cells the least, and the HT-29 between these two. In contrast, the normal cell lines were minimally impacted. They-HT expression paralleled these results: The cells with the highest 5-HT levels were most inhibited by TE, while the normal cell lines had very little to no 5-HT expression.

Telotristat ethyl is a potent inhibitor of tumor cell line growth. The efficacy of this inhibition varies between cell lines, appearing to be related to the levels of endogenous 5-HT. This suggests that TE may be most effective against tumors with high 5-HT production. Therefore, identification of 5-HT producing tumors may yield a list of TE targets.

### Example 2

### Telotristat Ethyl Combination Treatment in Tumor Xenograft Models

This example demonstrates the efficacy of telotristat ethyl in combination with cancer therapeutics in cancer cell line xenograft model. This study evaluated the anti-tumor activity of telotristat ethyl in combination with cisplatin (CIS) and gemcitabine (GEM) in two tumor cell line xenograft models of human BTC and CCA.

### Methods

Assays were performed on KKU-M213 and HuCCGT1 cells and tumor tissue. Total RNA was extracted using the RNeasy Mini Kit (Qiagen, Hilden, Germany) and used for cDNA synthesis with iScript Reverse Transcription Supermix for real-time quantitative polymerase chain reaction (RT-qPCR [BioRad]). TPH-1 and β-actin vehicle primers were from BioRad (PrimePCR™ PreAmp for SYBR® Green Assay: Human TPH-1 and Human ACTB). RT-qPCR was performed using QuantStudio 6 Flex System (Applied Biosystems, Waltham, Mass.). Each sample was run in triplicate, normalized to β-actin, and analyzed by the comparative CT method (Livak K J, Schmittgen T D. Methods 2001; 25(4): 402-408). HeLa cells were used as a baseline comparator to determine relative mRNA levels. KKU-M213 and HuCCT1 tumor cell lines were selected as models of a high- and low-expressing TPH-1 activity, respectively, to assess the impact of TPH-1 inhibition with telotristat ethyl on tumor growth in combination with GEM+CIS

Six-week-old female athymic nude mice (Charles River, San Diego, Calif.) were randomly assigned to 3 groups (n=3 per group):
telotristat ethyl 200 mg/kg twice daily (bid)+GEM 80 mg/kg every 3 days (q3d)×4+CIS 2 mg/kg every week (qw)×2
telotristat ethyl 200 mg/kg bid+GEM 80 mg/kg q3d×4+CIS 3 mg/kg qw×2
telotristat ethyl 200 mg/kg bid+GEM 80 mg/kg q3d×4+CIS 4 mg/kg qw×2.
   Telotristat ethyl was administered via oral gavage (PO); GEM and CIS were administered via intraperitoneal (IP) injections. Body weights were recorded thrice weekly after dose initiation, and the percentage changes in body weights were recorded

Seven-week-old female athymic nude mice (Charles River, San Diego, Calif.) were inoculated with 0.1 mL of a 50% Media/50% Matrigel® mixture containing a suspension of 5×106 cells/mouse of KKU-M213 or HuCCT1 tumor cells (JCRB, Ibaraki City, Osaka, Japan). Tumor volume (TV) was calculated as: TV (mm³)=(a×b2/2) where ‘b’ is the smallest diameter and ‘a’ is the largest diameter. 4 (KKU-M213) or 7 days later (HuCCT1), 40 mice with tumor sizes 61-129 mm3 (KKU-M213) or 75-117 mm3 (HuCCT1) were randomly assigned to 4 groups:
Vehicle (0.25% methylcellulose) bid
Vehicle bid+GEM 80 mg/kg q3d×4+CIS 3 mg/kg qw×2
telotristat ethyl 100 mg/kg bid+GEM 80 mg/kg q3d×4+CIS 3 mg/kg qw×2
telotristat ethyl 200 mg/kg bid+GEM 80 mg/kg q3d×4+CIS 3 mg/kg qw×2.

TVs and body weights were recorded at randomization and twice weekly thereafter. Clinical observations were made daily. Animals were euthanized at a mean TV ≥1,000 mm³ (KKU-M213) or when substantial tumor necrosis necessitated study end (HuCCT1).

Study protocols were approved by the Translational Drug Development Institutional Animal Care and Use Committee, and all procedures were performed in accordance with institutional guidelines.

The primary endpoint was tumor growth inhibition (TGI). Mean TGIs were calculated for day 21 (KKU-M213) and day 47 (HuCCT1), the final days all mice were on study, using the formula: TGI=[1−(Mean Treated_{(final)}−Mean Treated_{(Day 1)}/Mean Vehicle_{(final)}−Mean Vehicle_{(Day 1)}]×100%.

Time to mean terminal TV was calculated for the KKU-M213 model using the days postdose starting from the treatment initiation date to the date of group euthanasia. Differences in day 21 or day 47 TV were confirmed using 2-tailed Student t-tests with Welch's corrections to verify differences between each treatment group and vehicle as well as triplet combination therapy and the standard agent group. All statistical analyses were performed with Prism GraphPad®.

### Results

mRNA levels: TPH-1 mRNA levels, relative to HeLa cells, in KKU-M213 and HuCCT1 cell lines were 112.06 and 8.84. TPH-1 mRNA levels, relative to HeLa cells, in KKU-M213 and HuCCT1 tumors were 138.68 and 1.21.

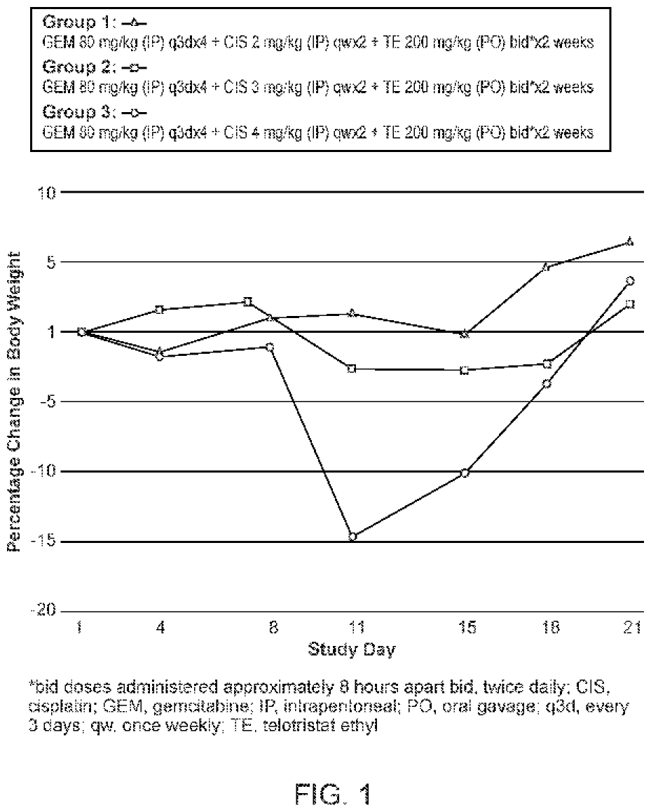

Tolerability: FIG. 1 provides the base effect of treatment on body weight for the athymic nude mouse model without xenographs. Groups 1 and 2 showed negligible (<5%) body weight loss during treatment. Group 3 showed maximum body weight loss (14%) by day 11. All body weights fully recovered by day 21. Based on these results, the 3-mg/kg dose of CIS was chosen for the xenograft studies.

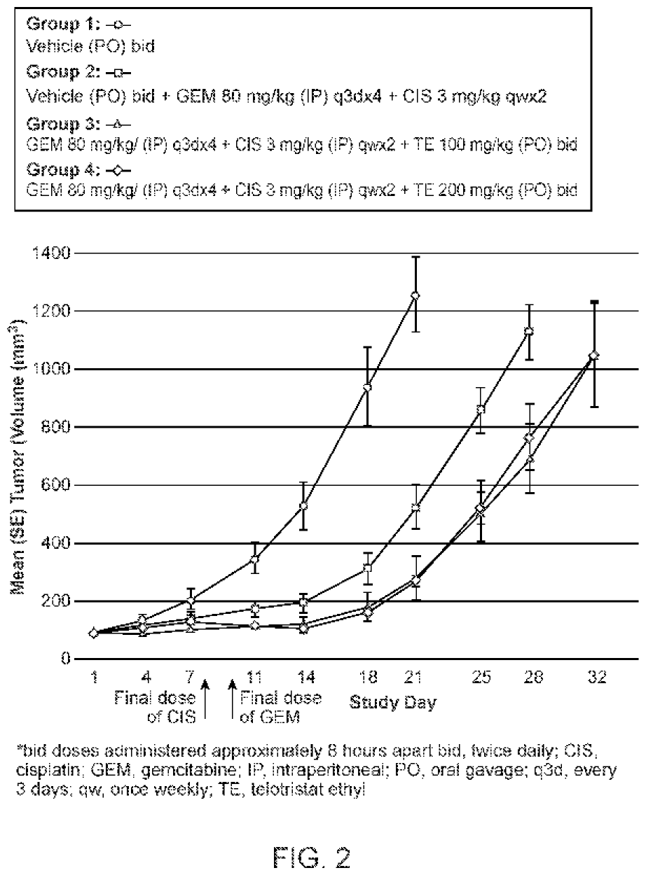

Xenograft Model KKU-M213: FIG. 2 provides the effect of treatment on tumor size for mice with KKU-M213 tumor cell line xenographs. Group 1 (vehicle) experienced no mean body weight loss; groups 2, 3, and 4 showed moderate weight loss with a maximum recorded on day 11; all body weights fully recovered to baseline values by day 18. Group 1 was terminated on day 21, group 2 on day 28, and groups 3 and 4 on day 32, when the mean TV was ≥1000 mm³.

Group 2 (GEM+CIS) demonstrated a 63% tumor growth inhibition (TGI) compared to vehicle (group 1), whereas groups 3 and 4 demonstrated a mean 83% TGI compared to vehicle (group 1) (Table 2-1). Similar inhibition patterns were observed for tumor volume (TV). In the KKU-M213 study, the vehicle group was euthanized on day 21 when the mean TV was ≥1,000 mm³ while all the groups in the HuCCT1 study were euthanized on day 57 before reaching ≥1000 mm³ due to substantial tumor necrosis throughout the vehicle group.

The difference in TV was statistically significant between groups 2, 3, and 4 and the vehicle group (p<0.05). The telotristat ethyl+GEM+CIS combinations significantly reduced TV compared to GEM+CIS alone (p<0.05). Groups 2, 3, and 4 remained on study longer than the vehicle group, and the telotristat ethyl+GEM+CIS combinations were treated longer than GEM+CIS alone. There was no difference in TGI or TV between the 2 telotristat ethyl doses (i.e., Groups 3 and 4).

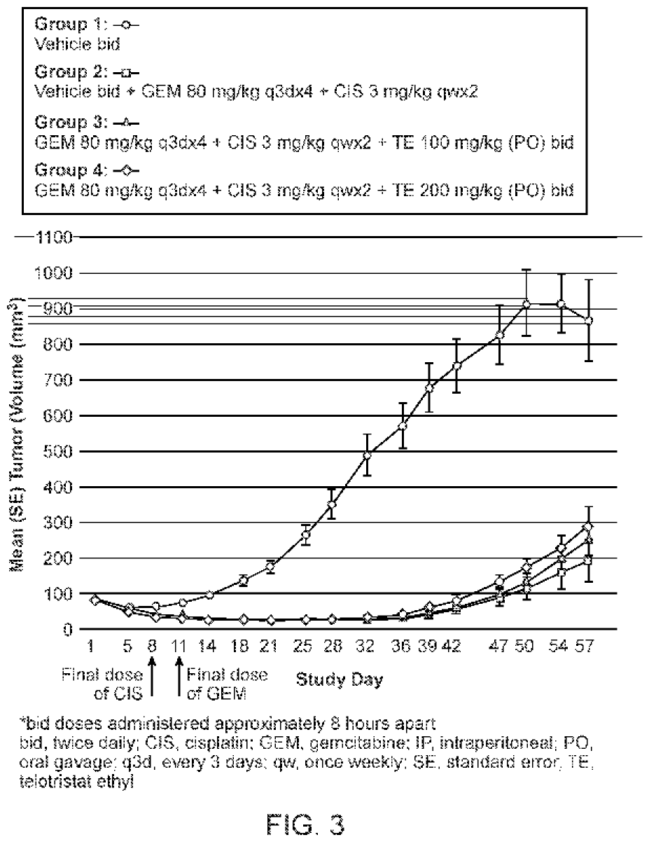

Xenograft Model HuCCT1: FIG. 3 provides the effect of treatment on tumor size for mice with HuCCT1 tumor cell line xenographs. Group 1 (vehicle) experienced no mean body weight loss; groups 2, 3, and 4 showed moderate weight loss with a maximum recorded on day 11; all body weights fully recovered by day 18. All groups were terminated on day 57 (before reaching TV ≥1000 mm³) owing to substantial tumor necrosis in the vehicle group. The difference in TV was statistically significant between groups 2, 3, and 4 and the vehicle group (p<0.001); however, the telotristat ethyl+GEM+CIS combination did not provide further reduction in TGI and TV versus GEM+CIS alone (Table 2-1).

TABLE 2-1
KKU-M213 and HuCCT1 Results
KKU-M213 Group

| | Time to | | | t-test | t-test |
|---|---|---|---|---|---|
| | Terminal | Day 21 | Day 21 TV | p-value | p-value |
| | TV | TGI | (mm³) Mean | vs. | Vehicle + |
| | (days) | (%) | (SE) | Vehicle | GEM + CIS |
| Vehicle | 20 | — | 1246.3 (129.3) | — | — |
| Vehicle + | 27 | 63.0 | 516.9 (78.0) | <0.05 | — |
| GEM + CIS | | | | | |
| TE100 + | 31 | 83.8 | 276.6 (78.3) | <0.05 | <0.05 |
| GEM + CIS | | | | | |
| TE200 + | 31 | 84.7 | 265.6 (23.3) | <0.05 | <0.05 |
| GEM + CIS | | | | | |

HuCCT1 Group

| | Time to | | | t-test | t-test |
|---|---|---|---|---|---|
| | Terminal | Day 47 | Day 47 TV | p-value | p-value |
| | TV | TGI | (mm³) Mean | vs. | Vehicle + |
| | (days) | (%) | (SE) | Vehicle | GEM + CIS |
| Vehicle | — | — | 825.4 (83.8) | — | — |
| Vehicle + | — | 99.0 | 96.4 (26.5) | <0.001 | — |
| GEM + CIS | | | | | |
| TE100 + | — | 98.0 | 103.5 (16.3) | <0.001 | Ns |
| GEM + CIS | | | | | |
| TE200 + | — | 93.3 | 138.2 (22.1) | <0.001 | Ns |
| GEM + CIS | | | | | |

a Vehicle bid
b Vehicle bid + GEM 80 mg/kg q3d × 4 + CIS 3 mg/kg qw × 2
c TE 100 mg/kg bid + GEM 80 mg/kg q3d × 4 + CIS 3 mg/kg qw × 2
d TE 200 mg/kg bid + GEM 80 mg/kg q3d × 4 + CIS 3 mg/kg qw × 2
bid, twice daily;
CIS, cisplatin;
GEM, gemcitabine;
ns, not significant;
q3d, every 3 days;
qw, once weekly;
SE, standard error;
TE, telotristat ethyl;
TV, tumor volume.

### Conclusions

In the KKU-M213 model, telotristat ethyl significantly reduced TV and growth rates and increased TGI when combined with GEM+CIS versus treatment with GEM and CIS alone. Similar antitumor effects were observed with telotristat ethyl at 100 and 200 mg/kg when combined with GEM and CIS. In the HuCCT1 model, the combination of telotristat ethyl with GEM+CIS did not improve the antitumor effects compared to treatment with GEM+CIS alone. Because TPH-1 mRNA levels were ˜115 times higher in KKU-M213 versus HuCCT1 tumors, it is postulated that the antitumor response observed in the KKU-M213 model may be related to high TPH-1 levels.

### Example 3

### Telotristat Ethyl Treatment in Patients with Neuroendocrine Tumors

Cholangiocarcinoma (CCA) has a poor prognosis with a 5-year survival rate of 5-15%. Gemcitabine plus cisplatin (GemCis) is the standard therapy for CCA. Nab-paclitaxel (NPT) is an approved treatment for breast, lung and pancreatic cancer. The therapeutic efficacy of telotristat ethyl (telotristat ethyl), an inhibitor of serotonin biosynthesis, was determined in combination with cytotoxic therapy in preclinical CCA models.

### Methods

The human intrahepatic CCA cells CCLP-1 were cultured in DMEM containing 10% FBS. Animal survival was studied in a peritoneal dissemination model, and a tumor growth study was performed in subcutaneous xenografts using 4-6 weeks old female NOD/SCID mice by injecting 10×106 CCLP-1 cells per mouse. Ten days after tumor cell injection, mice were randomized (n=4-7) to receive PBS (control), gemcitabine (50 mg/kg, 2×wk), cisplatin (2.5 mg/kg, 2×wk survival study, 1×wk tumor growth study), nab-paclitaxel (5 mg/kg, 2×wk) or telotristat ethyl (100 mg/kg, 5×wk), for two weeks. In the survival study, mice were monitored daily and euthanized when moribund, and survival was measured from the first day of treatment until death. In the tumor growth study, tumor size was measured twice per week and tumor volume was measured using the formula V=½ (L×W2, with L=length and W=width). Comparative statistics utilized one-way ANOVA, Student's t-test and logrank methods.

### Results

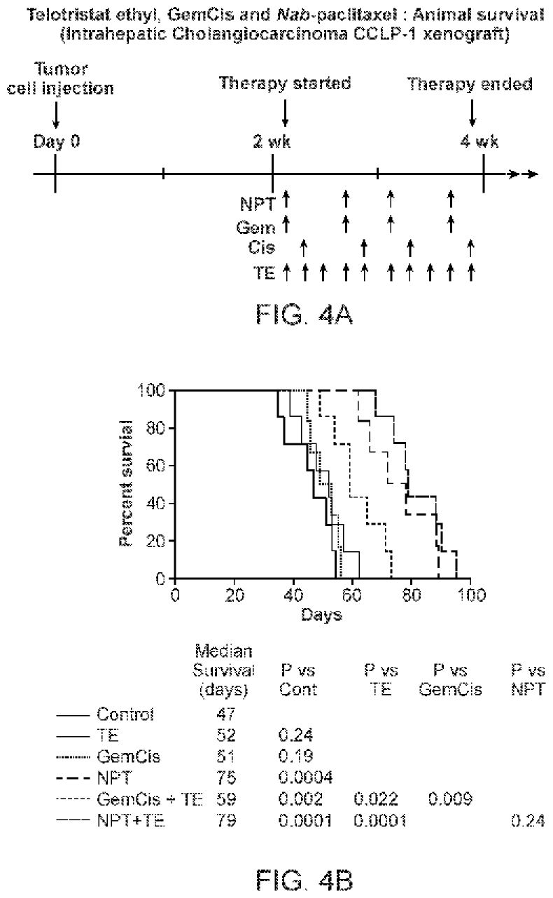

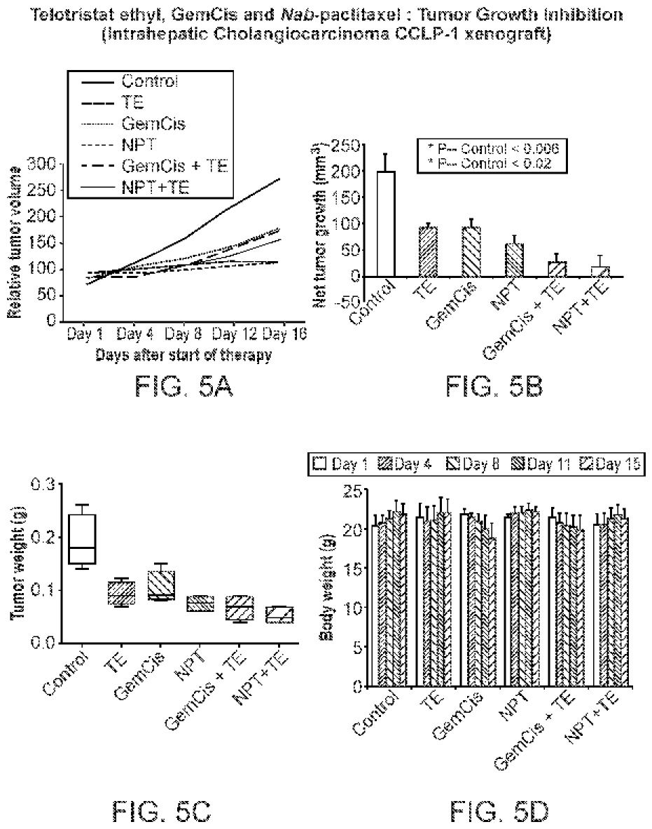

Animal survival was slightly increased by telotristat ethyl (52 days) or GemCis (51 days), compared with controls (47 days) (FIG. 4B). NPT led to a greater improvement in animal survival (75 days, a 59% increase) (FIG. 4B). Importantly, the combination of telotristat ethyl with GemCis or NPT demonstrated a significant increase in animal survival: GemCis+telotristat ethyl (59 days, a 26% increase) and NPT+telotristat ethyl (79 days, a 68% increase) (FIG. 4B). In subcutaneous xenografts, control tumors grew fast and therapy with telotristat ethyl, GemCis or NPT caused a reduction in tumor growth (FIGS. 5A-5C). Importantly, combinations of telotristat ethyl with cytotoxic agents exhibited an additive effect. In this study, compared to controls (199 mm³), the net tumor growth was 93 mm³ (telotristat ethyl), 94 mm³ (GemCis), 62 mm³ (NPT), 29 mm³ (GemCis+telotristat ethyl) and 19 mm³ (NPT+telotristat ethyl) (FIG. 5B). At the completion of therapy, tumor weight was maximum in controls (0.187 g) that was decreased by telotristat ethyl (0.091 g), GemCis (0.099 g) and NPT (0.074 g) (FIG. 5C). The addition of telotristat ethyl to chemotherapy further decreased the tumor weight: GemCis+telotristat ethyl (0.065 g) and NPT+telotristat ethyl (0.051 g) (FIG. 5C).

### Conclusion

Telotristat ethyl (TE) monotherapy showed antitumor activity in both in vivo model settings, and it also augmented antitumor effects of GemCis or NPT chemotherapy. The differential magnitude of antitumor activity of telotristat ethyl in the peritoneal dissemination and subcutaneous xenograft models can likely be attributed to possible differences in the tumor microenvironment between the two models. These findings provide a strong rationale for further evaluation of telotristat ethyl combinations with chemotherapy regimens (GemCis and NPT) to improve clinical outcomes for patients with advanced CCA.

TE, GemCis and NPT demonstrated a reduction in tumor growth in CCA subcutaneous xenografts. TE combinations with cytotoxic agents (GemCis or NPT) exhibited an additive effect on CCA tumor growth inhibition. TE and GemCis led to a smaller increase but NPT caused a greater increase in animal survival in CCA peritoneal dissemination xenografts. TE combinations with cytotoxic agents caused a further improvement in animal survival.

### Example 4

### Telotristat Ethyl Treatment in Patients with Neuroendocrine Tumors

This example provides a retrospective, single arm study of patients with locally advanced or metastatic neuroendocrine tumors (NETs) and documented carcinoid syndrome (CS). The patients received telotristat ethyl for at least 6 months. Telotristat ethyl showed substantial reductions in tumor size in the patients. This example reports progression-free survival (PFS), time to tumor progression, changes in carcinoid syndrome (CS) symptoms, and indictors of overall health.

Patients with poorly differentiated neuroendocrine tumors, mixed tumor types or conflicting clinical trial enrollment were excluded. Descriptive statistics, Kaplan-Meier and Chi-square tests were used to evaluate PFS, tumor progression, changes in CS symptoms, body weight and Eastern Cooperative Oncology Group (ECOG) performance status before and after telotristat ethyl initiation. Subgroup analyses were conducted in patients with the same pre- and post-telotristat ethyl background treatment.

Anonymized data for 200 patients were provided by 114 physicians; patients received telotristat ethyl for a median of nine months. Median time to tumor progression was 39.8 months (IQR, 18.7-39.8); most had no tumor progression at six (92%) and twelve months (87%) after telotristat ethyl initiation. Median progression-free survival was 23.7 months (17.8-39.8); most had progression-free survival at six (90%) and twelve months (80%). Results were consistent in the subgroup of 65 patients with the same pre/post-telotristat ethyl background treatment. Nearly all patients had improved carcinoid syndrome symptoms, stable or improved weight and Eastern Cooperative Oncology Group performance status after telotristat ethyl.

Patients with neuroendocrine tumors showed improvements in clinical outcomes and indicators of overall health following telotristat ethyl treatment, consistent with observed reductions in tumor size in this exploratory pilot study.

### Methods

Adults with unresectable locally advanced or metastatic neuroendocrine tumors and evidence of carcinoid syndrome documented in their medical chart were eligible for inclusion if they had received telotristat ethyl for at least 6 months and had any additional carcinoid syndrome and neuroendocrine tumor treatment information available for at least six months after telotristat ethyl initiation or until death. The presence of carcinoid syndrome was based on the participating physician's assessment of information contained in the medical charts. Participating physicians were recruited by a professional recruiting organization (Dynata, Plano, Tex., USA) and had to have treated at least one eligible patient with telotristat ethyl in the past 12 months in order to participate. Eligible records had to have documented tumor size and tumor response assessments before and after telotristat ethyl initiation, including at least two radiological scans in the 12 months before telotristat ethyl and at least one scan after telotristat ethyl initiation. Patients with a histologically poorly differentiated neuroendocrine tumor based on grade (G3) or Ki67 index >20%, mixed tumor types according to physician notes, or documented enrollment in any clinical trial during the 6 months after telotristat ethyl initiation were excluded.

Table 4-1 provides a summary of selected characteristics of the overall patient population in the study.

TABLE 4-1
Demographic and Clinical Characteristics of Patients

| Characteristic, n (%) unless noted | Patients (n = 200) | |
|---|---|---|
| Age at TE initiation, mean (SD) | 60.6 (10.2) | |
| Male | 113 (57) | |
| Race | | |
| White | 148 (74) | |
| Black or African American | 35 (18) | |
| Asian | 13 (7) | |
| Native American or American Indian | 3 (2) | |
| Unknown/not sure | 3 (2) | |
| Ethnicity | | |
| Hispanic | 24 (12) | |
| Non-Hispanic | 176 (88) | |
| NET histologic differentiation | | |
| Well differentiated | 122 (61) | |
| Moderately differentiated | 78 (39) | |
| Primary site of tumor | | |
| Pancreas | 52 (26) | |
| Small bowel | 35 (18) | |
| Lung, bronchus, larynx, trachea, | 19 (10) | |
| other respiratory organs | | |
| Stomach | 16 (8) | |
| Jejunum | 16 (8) | |
| Duodenum | 13 (7) | |
| Ileum | 13 (7) | |
| Appendix | 9 (5) | |
| Colon | 8 (4) | |
| NET of unknown primary origin | 8 (4) | |
| Small bowel mesentery | 7 (4) | |
| Cecum | 3 (2) | |
| Rectum | 1 (1) | |
| ECOG Performance Status | | |
| 0 | 61 (31) | |
| 1 | 111 (56) | |
| 2 | 26 (13) | |
| Unknown/not sure | 2 (1) | |
| | 33 (17) | |
| | 103 (52) | |
| | 61 (31) | |
| | Before TE, | After TE, |
| Patients receiving SSA treatment | (n = 95) | (n = 160) |
| Octreotide, long-acting | 66 (69) | 95 (59) |
| Lanreotide | 28 (29) | 51 (32) |
| Octreotide, short-acting or rescue | 5 (5) | 15 (9) |
| Pasireotide | 0 | 3 (2) |
| Patients receiving non-SSA NET | Before TE, | After TE, |
| treatment | (n = 35) | (n = 52) |
| Liver-directed therapy (non-surgical) | 12 (34) | 9 (17) |
| Surgery | 12 (34) | 13 (25) |
| Chemotherapy | 10 (29) | 15 (29) |
| Targeted therapy | 4 (11) | 13 (25) |
| Interferon | 2 (6) | 5 (10) |
| Other therapy^{b} | 2 (6) | 10 (19) |
| Radiological scans performed | (n = 450) | (n = 241) |
| CT | 256 (57) | 135 (56) |
| ⁶⁸Ga-DOTATOC SSTR PET | 78 (17) | 42 (17) |
| MRI | 62 (14) | 22 (9) |
| Other | 119 (26) | 68 (28) |

ECOG, Eastern Cooperative Oncology Group;
NET, neuroendocrine tumor;
SD, standard deviation;
SSA, somatostatin analog;
TE, telotristat ethyl.
The percentages may not sum to 100% due to multiple treatments per patient or due to rounding.
a Gastrointestinal tumor sites included: appendix, cecum, colon, duodenum, ileum, jejunum, rectum, small bowel, small bowel mesentery, and stomach
^{b}Other therapies included peptide-receptor radionuclide therapy (Lu-177), external beam radiation, and peptide-receptor radionuclide therapy (yttrium-90).

Participating physicians and study sponsor personnel were blinded to the identity of the other. A randomization scheme was implemented during chart abstraction where a random sequence of letters was generated to determine the selection of each medical chart for review; the letters were not retained or recorded. An automatically generated date shift (addition or subtraction of a randomly generated number of days) was assigned to each patient to further preserve the de-identification of collected data. This retrospective chart review maintained the anonymity of patients' medical records that were abstracted for review and analysis. New England Independent Review Board® (neirb.com) reviewed the study protocol and electronic case report form and determined the study to be exempt from IRB review.

### Outcomes

Time to tumor progression was defined as the time from telotristat ethyl initiation to the first documented tumor progression in the medical chart. Patients who did not experience tumor progression were censored at their last radiological scan. Progression-free survival (PFS) was defined as the time from telotristat ethyl initiation to tumor progression or death. Patients who did not experience tumor progression or death were censored at last follow-up. Physician-assessed changes in body weight, Eastern Cooperative Oncology Group (ECOG) Performance Status, and CS symptoms including diarrhea, flushing, abdominal pain, nausea and ascites were abstracted from medical charts before and after telotristat ethyl initiation.

### Statistical Analysis

In order to evaluate the effect of telotristat ethyl treatment on secondary clinical outcomes, analyses were conducted in the overall population and in a subgroup of patients who had the same documented non-telotristat ethyl neuroendocrine tumor treatment before and after telotristat ethyl initiation. Descriptive statistics summarized patient demographic and clinical characteristics, and changes in body weight and ECOG Performance Status after telotristat ethyl treatment initiation. Kaplan-Meier analyses estimated median time to tumor progression and median PFS. Chi-square test evaluated changes in documented CS symptoms (improved vs same/worsened) before and after telotristat ethyl initiation. All analyses were performed using SAS version 9.4 (SAS Institute, Cary, N.C., USA).

### Results

One hundred and fourteen physicians, predominantly oncologists (98%) from community practices (62%), provided anonymized, abstracted medical record data. Data were provided from a median of 1.0 (IQR, 1-2) individual patient charts per physician.

Of the 200 study patients, most had a gastrointestinal primary tumor site and well differentiated neuroendocrine tumors (both 61%). Patients received telotristat ethyl for a median of 9 months, and 82% were still receiving telotristat ethyl at the time of data collection. Sixty-five patients comprised a subgroup of those with the same NET treatment in the pre- and post-telotristat ethyl periods, with a median 7.7 months of telotristat ethyl treatment. This group was analyzed separately as well because of the potential to investigate the pure effect of telotristat ethyl therapy alone.

### Time to Tumor Progression and Progression Free Survival

The median time to tumor progression in the overall population was 39.8 months (IQR: 18.7, 39.8; Table 4-1). The majority of patients had no tumor progression at 6 months (92%), 12 months (87%) and 18 months (78%) after telotristat ethyl initiation. The median PFS was nearly 2 years at the time of data collection (23.7 months; IQR: 17.8, 39.8). Most patients had progression-free survival at 6 months (90%), 12 months (80%) and 18 months (72%) after telotristat ethyl initiation. Results for progression-free survival and time to tumor progression in the subgroup of patients with the same neuroendocrine tumor and carcinoid syndrome background treatments (n=65) were slightly more favorable but generally consistent with those of the overall population (Table 4-2).

TABLE 4-1
Time to tumor progression and progression-free survival
(Overall population)

| | | | |
|---|---|---|---|
| | | Patients | Patients without |
| Outcome | Events | at risk | outcome (%) |

Tumor progression

| | | | |
|---|---|---|---|
| 6 months | 12 | 83 | 92.0% |
| 12 months | 16 | 34 | 86.8% |
| 18 months | 18 | 13 | 78.3% |

Progression-free survival

| | | | |
|---|---|---|---|
| 6 months | 16 | 83 | 89.5% |
| 12 months | 23 | 34 | 79.9% |
| 18 months | 25 | 13 | 72.1% |

TABLE 4-2
Time to tumor progression and progression-free survival
(Treatment subgroup, n = 65)

| | | | |
|---|---|---|---|
| | | Patients | Patients without |
| Outcome | Events | at risk | outcome (%) |

Tumor progression

| | | | |
|---|---|---|---|
| 6 months | 0 | 27 | 100.0% |
| 12 months | 2 | 7 | 90.4% |
| 18 months | 2 | 5 | 90.4% |

Progression-free survival

| | | | |
|---|---|---|---|
| 6 months | 1 | 27 | 96.9% |
| 12 months | 3 | 7 | 87.5% |
| 18 months | 3 | 5 | 87.5% |

### Tumor Size Pre-Telotristat Ethyl Initiation and Post-Telotristat Ethyl Initiation

A radiological scan performed immediately before telotristat ethyl initiation was evaluated. A radiological scan performed post-telotristat ethyl was also examined. Paired t-test was used to calculate P-value for tumor size. Statistical significance (P<0.001) was also confirmed using Wilcoxon signed rank test. An average (SD) tumor size reduction of 0.5 (1.8) cm from the radiological scan performed immediately before telotristat ethyl initiation until the last radiological scan performed post-telotristat ethyl was observed (P<0.001).

TABLE 4-3
Tumor Size Pre- and Post-TE Initiation (N = 200)

| | Pre-TE | Post-TE | Change from | |
|---|---|---|---|---|
| Tumor | Initiation | initiation | baseline | |
| size (cm) | (N = 200) | (N = 200) | (N = 200) | P-value |
| Mean ± SD | 3.9 ± 2.2 | 3.4 ± 2.3 | −0.5 ± 1.8 | <0.001* |
| Mean (IQR) | 3.5 (2.3, 5.0) | 3.0 (2.0, 4.8) | — | |

*Statistically significant at p < 0.05.

### Longitudinal Analysis of Tumor Growth (%)

Generalized linear regression with a log link and the generalized estimating equations method was used. Time of the first tumor scan during pre-telotristat ethyl period was used as baseline. Negative estimate indicates a decrease in tumor size after telotristat ethyl initiation. An estimate of −8.5% represents an 8.5% decrease in tumor size during the period after telotristat ethyl initiation. The Wald test P-values from the regression model are reported. An 8.5% decrease in tumor size (P=0.045) during the period after telotristat ethyl initiation was observed.

TABLE 4-4
Longitudinal Analysis of Tumor Growth

| Parameter | Estimate | 95% CI | P-value |
|---|---|---|---|
| Main Treatment Effect | | | |
| Difference in tumor size | −8.5% | (−16.1%, −0.2%) | 0.045* |
| between pre and post-TE | | | |
| initiation | | | |
| Covariates | | | |
| Time since first scan (months) | 0.5% | (0.0%, 1.1%) | 0.057 |
| SSA prior to TE initiation | −7.6% | (19.0%, 5.3%) | 0.235 |
| Additional NET treatment prior | 9.4% | (−8.9%, 31.5%) | 0.335 |
| to TE initiation | | | |

*Statistically significant at P < 0.05;
TE telotristat ethyl;
SSA somatostatin analog;
NET neuroendocrine tumor.

### Physician Assessed Tumor Response Post-Telotristat Ethyl Initiation Grouped by Tumor Response Pre-Telotristat Ethyl Initiation

Physician assessed tumor responses from radiological scans pre-telotristat ethyl initiation and post-telotristat ethyl initiation were included in the analysis. Three patients with “Other” tumor response (i.e., “worsened health”, “minor increase”, or “unknown assessment”) pre-telotristat ethyl initiation were excluded. Among 33 patients who had worsened (progressed) tumor response during pre-telotristat ethyl initiation period, 45% had improved (responded completely or partially) tumor response during post-telotristat ethyl initiation period.

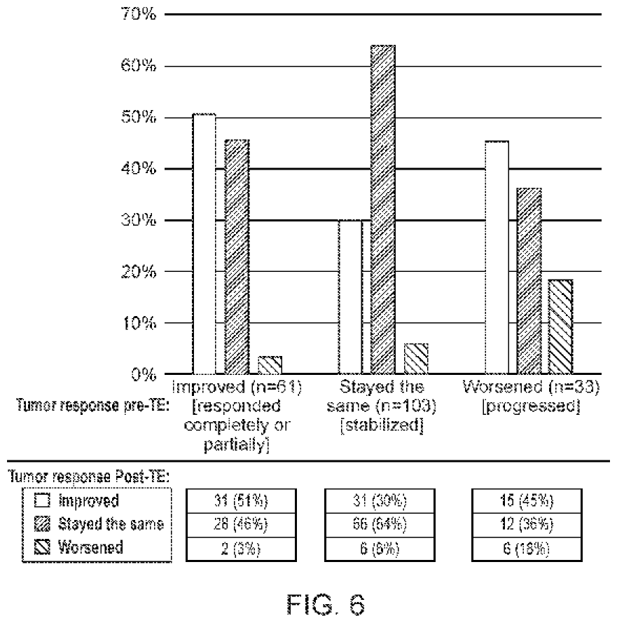

As shown in FIG. 6, most patients who had improved or stayed the same (stabilized) tumor response during pre-telotristat ethyl initiation period reported improved or stayed the same tumor response during post-telotristat ethyl initiation period.

### Kaplan Meier Estimates of Time to Tumor Progression and Progression Free Survival Post-Telotristat Ethyl Initiation

Time to tumor progression (TTP) and progression-free survival (PFS) were estimated by the Kaplan-Meier method. Median TTP was 39.8 months (IQR: 18.7, 39.8); median PFS was 23.7 months (IQR: 17.8, 39.8). By 18 months, approximately 78% did not experience tumor progression, and approximately 72% of patients had PFS.

TABLE 4-5
Time to Tumor Progression and Progression-Free Survival Estimates

| | TTP | | | PFS | | |
|---|---|---|---|---|---|---|
| | | | % Patients | | | % Patients without |
| Time after | | | without tumor | | | tumor progression or |
| TE Initiation | N events | N at risk | progression | N events | N at risk | death |
| 6 months | 12 | 83 | 92.0% | 16 | 83 | 89.5% |
| 12 months | 16 | 34 | 86.8% | 23 | 34 | 79.9% |
| 18 months | 18 | 13 | 78.3% | 25 | 13 | 72.1% |

### Changes in Carcinoid Syndrome Symptoms, Body Weight and ECOG Performance Status

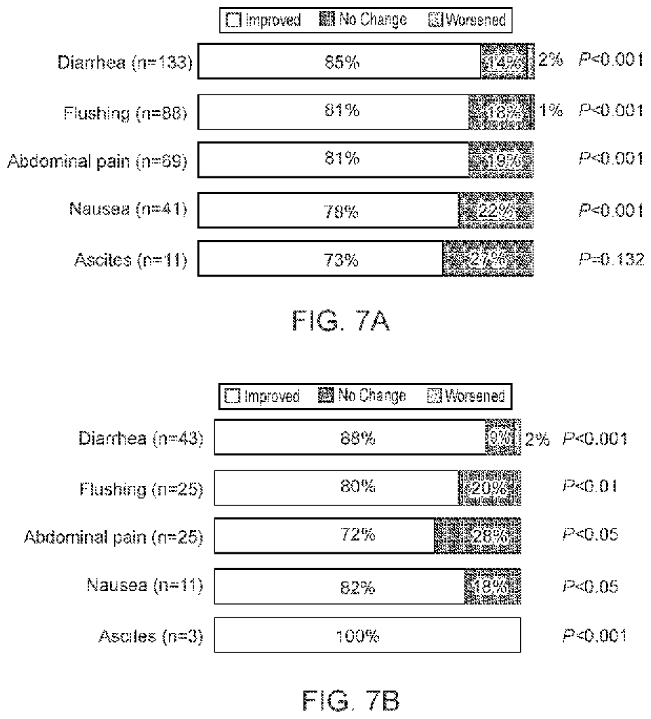

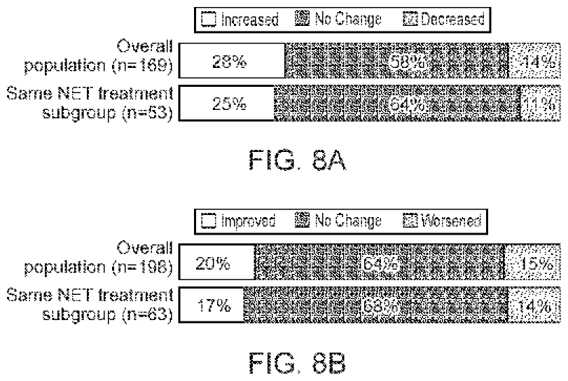

The majority of patients had improved carcinoid syndrome symptoms in the post-telotristat ethyl period, both in the overall population and in the subgroup of patients with the same neuroendocrine tumor treatment before and after telotristat ethyl (shown in FIGS. 7A and 7B). Body weight and ECOG Performance Status were improved or unchanged for most patients in both the overall population and the same NET treatment subgroup (shown in FIGS. 8A and 8B).

### Discussion

This example presents clinical outcomes from the TELEACE study, which showed reductions in tumor size with telotristat ethyl independent of background neuroendocrine tumor treatment. Positive outcomes were observed related to tumor progression and progression-free survival along with improved clinical outcomes in patients with advanced neuroendocrine tumors receiving telotristat ethyl in US clinical practice. Most patients had no tumor progression and had progression-free survival through 18 months of follow-up after starting telotristat ethyl. Reductions in average tumor size were observed after adjusting for treatment received prior to telotristat ethyl. Improvement in physician assessed tumor response was observed in the majority of patients following telotristat ethyl initiation. Patients showed significant improvements in carcinoid syndrome symptoms and clinical indicators of overall health after an average of 12 months of telotristat ethyl treatment. Findings were consistent for the overall population and a subgroup of patients with the same neuroendocrine tumor treatments before and after telotristat ethyl initiation, to ascertain the effect of telotristat ethyl treatment alone. At the time of telotristat ethyl initiation, the majority of patients were classified as having stable or improving tumor status by physician assessment, which was consistent with the expected use of telotristat ethyl in clinical practice. The finding that telotristat ethyl may have antiproliferative effects and improve clinical outcomes is notable in this setting.

### Example 5

### Biomarker Effects of Telotristat Ethyl Treatment of Neuroendocrine Tumors

This example provides the effects of telotristat ethyl treatment on selected patient biomarkers (i.e., u5-HIAA; CgA; 5-HT; and NT-proBNP) in the retrospective, single arm study of Example 4. It also shows the relationship between the change in biomarker values and the change in tumor progression between the pre- and post-TE periods.

### Methods

The average (SD) duration of documented TE treatment was 12.0 (7.3) months with a median (IQR) of 9.0 (6.8, 15.2) months. In many cases, a patient received a TE dosage of 250 mg three times per day.

To quantify changes in laboratory biomarkers, the time (in months) of biomarker assessments to/since TE initiation were determined using mean, standard deviation (SD), median, and interquartile range (IQR).

The continuous measures of biomarker values pre- and post-TE initiation were described using mean, SD, median, and IQR. The biomarkers were assessed only among patients with complete biomarker assessment results in both pre-TE and post-TE initiation periods. A Wilcoxon signed rank test was used to calculate P-values for the mean change in biomarker value.

Patients with reported biomarker values below, at, or above normal range in pre-TE and post-TE initiation periods were described using proportions and frequencies. The biomarkers were assessed only among patients with complete biomarker assessment results in both pre-TE and post-TE initiation periods. McNemar's test was used to calculate P-values for the differences in paired proportions of patients with values above the upper limit of normal.

To evaluate the relationship between the change in biomarker values (i.e., u5-HIAA; CgA; 5-HT) and the change in tumor progression between the pre- and post-TE periods, Pearson's correlation coefficient was used to assess the correlation between biomarker assessments and tumor sizes and to derive the associated P-values. The correlations were assessed only among patients with complete biomarker assessment results in both pre-TE and post-TE initiation periods. The relationship between biomarker values and tumor sizes during pre-TE and post-TE initiation period was presented using scatterplots.

To evaluate the change in CS symptoms, weight, and ECOG performance status between the pre- and post-TE periods, patients with documented CS symptoms (i.e., abdominal pain, ascites, diarrhea, flushing, nausea) pre-TE initiation were described using proportions and frequencies. The change in documented CS symptoms, weight, and ECOG performance status after TE Initiation (i.e., improved/increased, stayed the same/remained unchanged, worsened/decreased) were also described using proportions and frequencies. P-values were calculated using Chi-square test for equal proportions for change in CS symptoms (i.e., proportion of patients with improved vs. stayed the same/worsened CS symptoms).

### Results

For the biomarker results below, the pre-TE initiation period is defined as 12 months before the index date (initiation of TE). The assessment closest to the index date during the pre-TE period was reported for each biomarker.

The post-TE initiation period is defined as a period from index date to the earlier of end of treatment with TE or date of chart abstraction. The assessment closest to the date of chart abstraction during the post-TE period was reported for each biomarker.

The data includes patients with reported biomarker values, units, and laboratory ranges during pre- and post-TE initiation periods. It excludes patients who had different biomarker units or laboratory ranges reported pre- and post-TE initiation.

The normal range was defined by reported laboratory lower limit of normal and upper limit of normal.

Level of u5 HIAA: As shown in Table 5-1 below, a significant reduction in mean u5-HIAA (n=22) was observed among patients with u5-HIAA pre- and post-TE initiation.

TABLE 5-1
Changes in Lab Biomarkers After TE Initiation—u5-HIAA

| | Pre-TE | Post-TE | |
|---|---|---|---|
| | Initiation | Initiation | |
| u5-HIAA | N = 44 | N = 32 | P-value |
| Time to/since TE initiation | | | |
| (months) | | | |
| Mean ± SD | 5.3 ± 4.7 | 6.6 ± 6.2 | — |
| Median (IQR) | 4.0 (1.0, 11.0) | 5.3 (1.0, 10.0) | — |
| Patients with biomarker | 22 | 22 | — |
| assessments in both | | | |
| periods, n | | | |
| Measurement (mg/24 | | | |
| hours) | | | |
| Mean ± SD | 72.5 ± 98.0 | 43.6 ± 54.7 | <0.001 |
| Median (IQR) | 42.0 (25.0, 65.0) | 25.0 (16.0, 44.0) | — |
| Biomarker value, n (%) | | | |
| Below normal range | 0 (0) | 0 (0) | — |
| At normal range | 1 (4.5) | 2 (9.1) | — |
| Above normal range | 21 (95.5) | 20 (90.9) | 0.317 |

The values in Table 5-1 have been converted to mg/24 hours based on the following conversion rate: 1 μmol/24 hours=5.23 mg/24 hours. The Wilcoxon signed rank test was used to calculate P-values for the mean change in biomarker values pre- and post-TE initiation.

Level of 5-HT: As shown in Table 5-2 below, a significant reduction in mean 5-HT (n=22) was observed among patients with 5-HT pre- and post-TE initiation.

TABLE 5-2
Changes in Lab Biomarkers After TE Initiation—5-HT

| | Pre-TE | Post-TE | |
|---|---|---|---|
| | Initiation | Initiation | |
| 5-HT | N = 41 | N = 32 | P-value |
| Time to/since TE initiation | | | |
| (months) | | | |
| Mean ± SD | 6.2 ± 4.2 | 8.3 ± 5.1 | — |
| Median (IQR) | 7.4 (2.1, 9.9) | 8.3 (4.9, 9.5) | — |
| Patients with biomarker | 22 | 22 | — |
| assessments in both | | | |
| periods, n | | | |
| Measurement (ng/24 | | | |
| hours) | | | |
| Mean ± SD | 648.9 ± 448.1 | 533.5 ± 329.2 | 0.026 |
| Median (IQR) | 459.5 (389.0, | 399.5 (320.0, | — |
| | 701.0) | 700.0) | |
| Biomarker value, n (%) | | | |
| Below normal range | 0 (0) | 0 (0) | — |
| At normal range | 1 (4.5) | 2 (9.1) | — |
| Above normal range | 21 (95.5) | 20 (90.9) | 0.317 |

The Wilcoxon signed rank test was used to calculate P-values for the mean change in biomarker values pre- and post-TE initiation.

Level of CgA: As shown in Table 5-3 below, almost 30% of patients with CgA value above normal range during pre-TE initiation period had normal CgA value post-TE initiation.

TABLE 5-3
Changes in Lab Biomarkers After TE Initiation—CgA

| | Pre-TE | Post-TE |
|---|---|---|
| | Initiation | Initiation |
| CgA | N = 42 | N = 30 |
| Time to/since TE initiation | | |
| (months) | | |
| Mean ± SD | 6.7 ± 4.0 | 4.3 ± 4.2 |
| Median (IQR) | 6.1 (3.7, 10.5) | 3.1 (0.9, 7.3) |
| Patients with biomarker | 15 | 15 |
| assessments in both periods, n | | |
| Biomarker value, n (%) | | |
| Below normal range | 0 (0) | 0 (0) |
| At normal range | 0 (0) | 4 (26.7) |
| Above normal range | 15 (100.0) | 11 (73.3) |

McNemar's test could not be performed because all patients had CgA value above normal range pre-TE initiation.

Correlation Analysis Between Biomarker Assessments and Tumor Sizes During Pre-TE and Post-TE Initiation Period: As shown in Table 5-4 below, no clear correlations was observed between (1) biomarker and tumor size in the pre-TE period, (2) biomarker and tumor size in the post-TE period, and (3) change in the biomarkers and the change in tumor size.

TABLE 5-4
Correlation Analysis Between Biomarker Assessments and Tumor Sizes

| | Tumor Size (cm) | |
|---|---|---|
| Biomarker Assessment | Correlation | P-value |
| Pre-TE Initiation | | |
| u5-HIAA (mg/24 hours) | 0.28 | 0.204 |
| 5-HT (ng/mL) | 0.26 | 0.245 |
| Post-TE Initiation | | |
| u5-HIAA (mg/24 hours) | 0.36 | 0.099 |
| 5-HT (ng/mL) | 0.06 | 0.808 |
| | Mean Change in Tumor Size (cm) | |
| Biomarker value, n (%) | Correlation | P-value |
| u5-HIAA (mg/24 hours) | −0.15 | 0.501 |
| 5-HT (ng/mL) | −0.05 | 0.809 |

Table 5-4 is based on patients who had complete biomarker assessment results (u5-HIAA: N=22; 5-HT: N=22) in both pre-TE and post-TE initiation periods. Correlation analysis was not conducted for CgA and NT-proBNP due to small sample sizes for CgA (n=15) and NT-proBNP (n=2). Pearson's correlation coefficient was used to assess the correlation between biomarker assessments and tumor sizes and to derive the associated P-values. Values have been converted to mg/24 hours based on the following conversion rate: 1 μmol/24 hours=5.23 mg/24 hours.

Change in Documented Carcinoid Syndrome Symptoms of Patients from Medical Charts after TE Initiation: After TE initiation, the majority of patients experienced significant improvement in diarrhea (85%, n=133), flushing (81%, n=88), abdominal pain (81%, n=69) and nausea (78%, n=41) compared to same/worsened symptoms (FIG. 7A). Similar improvements were also observed in a patient subgroup with the same NET treatment before and after TE (FIG. 7B). The symptoms described did not include urgency or fecal incontinence although these symptoms may have been present. The Chi-square test P-values for equal proportions (i.e., proportion of patients with improved vs. stayed the same/worsened CS symptoms) were also reported.

### Example 6

### Efficacy of Telotristat Ethyl (TE) in Patients with Progressive Neuroendocrine Tumor Disease: Real-World Clinical Practice Experience

A follow-up analysis of real-world clinical practice experience with TE was initiated in patients with physician-documented worsening tumor status based on radiological reports. The analysis was directed to the subgroup of patients from the TELEACE study with worsening tumor status at the time of TE initiation. Baseline carcinoid syndrome (CS) symptoms, Eastern Cooperative Oncology Group (ECOG) performance status, and weight were compared after at least six months of TE treatment.

A total of 33 patients (17%) in TELEACE had worsened tumor status at the time TE of initiation. The mean patient age was 60 years, with 58% female and 76% white. The majority of these patients had intermediate grade tumor (67%). Prior to TE initiation, 61% had received SSA treatment, and 33% had received other NET treatment, which included liver-directed therapy, surgery, chemotherapy, targeted therapy, and Peptide Receptor Radionuclide Therapy (PRRT). The average duration of TE treatment was 14±10.5 months. Compared to baseline measures, each patient experienced improvement in at least one CS symptom. Additionally, a majority of patients had significant improvement/remained stable in CS symptoms, ECOG performance status, and weight after TE initiation (Table 6).

TABLE 6
Physician Assessed Outcomes Post TE initiation

| Outcomes | Improved | Remained the Same | P value |
|---|---|---|---|
| CS Symptoms | | | |
| Diarrhea (n = 26) | 88% | 12% | <0.001 |
| Flushing (n = 14) | 82% | 18% | 0.008 |
| Abdominal pain (n = 8) | 89% | 11% | 0.020 |
| Nausea (n = 4) | 80% | 20% | 0.432 |
| Weight (n = 31) | 32% | 55% | 0.017 |
| ECOG score (n = 33) | 24% | 73% | <0.001 |

In summary, the use of TE in patients with progressive NET demonstrated improvements in CS symptoms, functional status, and weight after TE initiation in most patients.

All publications and patent, applications cited in this specification are herein incorporated by reference as if each individual publication or patent application were specifically and individually indicated to be incorporated by reference. While the claimed subject matter has been described in terms of various embodiments, the skilled artisan will appreciate that various modifications, substitutions, omissions, and changes may be made without departing from the spirit thereof. Accordingly, it is intended that the scope of the subject matter limited solely by the scope of the following claims, including equivalents thereof

What is claimed:

1. A method of treating cancer in a patient in need thereof, comprising the step of administering to the patient a therapeutically effective amount of a tryptophan hydroxylase 1 (TPH1) inhibitor, wherein the TPH1 inhibitor is telotristat, a telotristat prodrug, or a pharmaceutically acceptable salt thereof.

2. The method of claim 1, wherein the TPH1 inhibitor reduces the growth rate of a neuroendocrine tumor in a patient.

3. The method of claim 2, wherein the neuroendocrine tumor expresses high levels of TPH1 activity relative to reference cells.

4. The method of claim 2, the method further comprising:
screening the neuroendocrine tumor for TPH1 messenger ribonucleic acid (mRNA) level; and
approving the patient for TPH1 inhibitor treatment only when the TPH1 mRNA level of the neuroendocrine tumor is at least 30 times that of the reference cells.

5. The method of claim 4, wherein the TPH1 mRNA level is at least 60 times that of the reference cells.

6. The method of claim 4, wherein the TPH1 mRNA level is at least 100 times that of the reference cells.

7. The method of claim 4, wherein the reference cells are HeLa cells.

8. The method of claim 2, the method further comprising:
screening the neuroendocrine tumor by a Ki-67 index test; and
approving the patient for TPH1 inhibitor treatment only when the Ki-67 index is no greater than 20%.

9. The method of claim 8, wherein the method comprises approving the patient for TPH1 inhibitor treatment only when the Ki-67 index is no greater than 3%.

10. The method of claim 8, wherein the method comprises approving the patient for TPH1 inhibitor treatment only when the Ki-67 index is no greater than 1%.

11. The method of claim 1, further comprising concurrently treating the patient with an antitumor agent.

12. The method of claim 11, wherein the antitumor agent is gemcitabine, cisplatin, paclitaxel, a paclitaxel derivative, or a combination thereof.

13. The method of claim 12, wherein the antitumor agent is a combination of gemcitabine and cisplatin.

14. The method of claim 12, wherein the antitumor agent is a combination of albumin and paclitaxel.

15. The method of claim 12 wherein the antitumor agent is nab-paclitaxel.

16. The method of claim 1, further comprising concurrently treating the patient with a somatostatin analog (SSA).

17. The method of claim 1, wherein the telotristat prodrug is telotristat ethyl.

18. The method of claim 1, wherein the pharmaceutically acceptable salt is a hippurate.

19. The method of claim 1, wherein the TPH1 inhibitor is telotristat ethyl hippurate.

20. The method of claim 1, where the cancer is biliary tract cancer, bladder cancer, breast cancer, prostate cancer, small-cell lung cancer, or a combination thereof.

21. The method of claim 20, where the biliary tract cancer is cholangiocarcinoma.

22. The method of claim 1, where the patient experiences carcinoid-syndrome-related diarrhea.

23. The method of claim 1, where the patient does not experience carcinoid-syndrome-related diarrhea.

24. The method of claim 1, wherein the TPH1 inhibitor dose is from 100 to 500 mg.

25. The method of claim 1, wherein the TPH1 inhibitor dose is about 250 mg.

26. The method of claim 1, wherein the TPH1 inhibitor dose is administered from one to five times per day.

27. The method of claim 1, wherein the TPH1 inhibitor dose is administered three times per day.
